(19) United States
(12) Patent Application Publication
(10) Pub. No.: US 2023/0263182 A1
(43) Pub. Date: Aug. 24, 2023

(54) COMPOSITIONS ISOLATED FROM DATE PALM TREE

(71) Applicant: The State of Israel, Ministry of Agriculture & Rural Development, Agricultural Research Organization, Rishon-Lezion (IL)

(72) Inventors: Hinanit KOLTAI, Rishon-LeZion (IL); Dvora NAMDAR, Beit Gamliel (IL)

(73) Assignee: The State of Israel, Ministry of Agriculture & Rural Development, Agricultural Research Organization, Rishon-Lezion (IL)

(21) Appl. No.: 18/024,075

(22) PCT Filed: Sep. 1, 2021
(86) PCT No.: PCT/IL2021/051075

§ 371 Date: Mar. 1, 2023

Related U.S. Application Data
(60) Provisional application No. 63/072,969, filed on Sep. 1, 2020.
(60) Provisional application No. 63/221,099, filed on Jul. 13, 2021.

(51) Int. Cl. A23D 9/04; C07D 239/22; B01D 11/04

(52) U.S. Cl. CPC A23D 9/04; C07D 239/22; B01D 11/0492

(57) ABSTRACT

Described herein are a method of preparing a vegetable oil, comprising pressing a non-fruit tissue of a date palm offshoot, as well as a vegetable oil and a polysaccharide-containing fraction obtained by such a method. Further described herein is an oil comprising esters of fatty acids, wherein at least 10 weight percent of the fatty acids are linolelaidic acid, as well as compositions comprising a variety of ingredients, such as saccharides and amino acids, as characterized herein, and a method of isolating dihydrouracil from an oil and/or composition described herein. Further described is a method of preparing a vegetable oil, comprising pressing date kernel tissue.

### RELATED APPLICATION(S)

This application claims the benefit of priority of U.S. Provisional Patent Application No. 63/072,969 filed on Sep. 1, 2020, and U.S. Provisional Patent Application No. 63/221,099 filed on Jul. 23, 2021, the contents of each of which are incorporated by reference as if fully set forth herein in their entirety.

### FIELD AND BACKGROUND OF THE INVENTION

The present invention, in some embodiments thereof, relates to utilization of natural products, and more particularly, but not exclusively, to isolation of compositions from palm trees.

“Palm oil” generally refers to an edible oil derived from the mesocarp (reddish pulp) of the fruit of oil palms, primarily the African oil palm (Elaeis guineensis), and to a lesser extent from the American oil palm (Elaeis oleifera) and the maripa palm (Attalea maripa). Palm oil is typically rich in palmitic acid and oleic acid, in somewhat equal proportions. The kernels of the fruit may also be used as a source of oil, which is often referred to as “palm kernel oil”. Palm kernel oil, like coconut oil (another oil derived from a palm species), is rich in short saturated fatty acids, particularly lauric and myristic acid.

The date palm is a tall, evergreen tree (Phoenix dactylifera L.) whose provenance for millennia is in the dry regions of the Middle East, Northern Africa, the Sahara and the Indus Valley. In contrast with other palm species, the date palm is characterized by its edible fruit, which has a high sugar content, and is primarily grown for food. According to reports in the scientific literature, oil has been extracted from date kernels of different date varieties using Soxhlet extraction, maceration, extraction with supercritical CO₂, or ultrasonication, with reported yields ranging from 3 to 14% [Mrabet et al., Foods 2020, 9:787]. The oil of date kernels has been found adequate for cosmetic uses, but the date kernels are usually not exploited for their relatively low oil content.

Date farming results in considerable amount of biomass as waste material. A date palm tree produces multiple cloned offshoots per year (e.g., 3-7 depending on its sub-type). In order to enhance fruit growth, the offshoots are commonly removed by growers to allocate the tree's energy from the offshoots to the fruits. Such offshoots are sometimes re-planted in order to renew and enlarge the date palm population, but usually they are discarded by burning.

Additional background art includes Namdar et al. [Archaeometry 2018, 60:571-593].

### SUMMARY OF THE INVENTION

According to an aspect of some embodiments of the invention, there is provided a method of preparing a vegetable oil having a desirable composition, the method comprising pressing agricultural waste derived from a date tree.

According to an aspect of some embodiments of the invention, there is provided a method of preparing a vegetable oil, the method comprising pressing a non-fruit tissue of a date palm offshoot, to thereby obtain the vegetable oil.

According to an aspect of some embodiments of the invention, there is provided a vegetable oil obtained according to the method of preparing a vegetable oil according to any of the embodiments described herein.

According to an aspect of some embodiments of the invention, there is provided a composition comprising a polar phase and a nonpolar phase, the composition being obtained according to the method of preparing a vegetable oil according to any of the embodiments described herein, wherein the nonpolar phase comprises the vegetable oil, and the polar phase comprises polysaccharides.

According to an aspect of some embodiments of the invention, there is provided a polysaccharide-containing fraction obtained according to any of the embodiments described herein relating to a method comprising separating a polysaccharide-containing fraction.

According to an aspect of some embodiments of the invention, there is provided an oil comprising esters of fatty acids, wherein at least 10 weight percent of the fatty acids are linolelaidic acid.

According to an aspect of some embodiments of the invention, there is provided a composition comprising asparagine, isoleucine, threonine, pipecolic acid, 5-hydroxypipecolic acid, allose, arabinose, galactose, ribose, talose, myo-inositol, linolenic acid, stearic acid, stearyl alcohol, and dihydrouracil. According to some such embodiments, the composition further comprises alanine, aspartate, glutamate, proline, serine, valine, N-methyl-leucine, xylose, glycerol, and palmitic acid.

According to an aspect of some embodiments of the invention, there is provided a composition comprising 5-hydroxypipecolic acid, hydroxyisocaproic acid, fructose, galactose, lyxose, psicose, rhamnose, ribose, sorbose, talose, trehalose, xylose, arabinoic acid, malic acid, citric acid, and kaur-16-ene. According to some such embodiments, the composition further comprises norvaline, arabinose, glucose, maltose, mannose, sucrose, and palmitic acid. According to some of any of the aforementioned embodiments of the invention, the composition further comprises esters of fatty acids, wherein at least 80 weight percent of the fatty acids are palmitic acid. According to some such embodiments, the fatty acids further comprise linolenic acid and stearic acid.

According to an aspect of some embodiments of the invention, there is provided a method of preparing a vegetable oil, the method comprising pressing date kernel tissue, to thereby obtain the vegetable oil.

According to an aspect of some embodiments of the invention, there is provided a method of obtaining a vegetable oil characterized by:
at least one of:
(a) at least 5 weight percent of the fatty acids of the oil are lauric acid;
(b) at least 5 weight percent of the fatty acids of the oil are myristic acid; and
(c) at least 8 weight percent of the fatty acids of the oil are lauric acid or myristic acid, as well as at least one of:
(i) at least 25 weight percent of the fatty acids of the oil are palmitic acid; and
(ii) no more than 30 weight percent of the fatty acids of the oil are oleic acid,
the method comprising combining:
an oil obtained from a non-fruit tissue of a date palm offshoot according to any of the respective embodiments described herein and/or comprising at least 10 weight percent linolelaidic acid according to any of the respective embodiments described herein, and
a vegetable oil from date kernel tissue.

According to an aspect of some embodiments of the invention, there is provided a method of obtaining a vegetable oil from agricultural waste material, the method comprising:
obtaining an oil from a non-fruit tissue of a date palm offshoot;
obtaining an oil from date kernel tissue; and
combining the oil from a non-fruit tissue of a date palm offshoot and the oil from date kernel tissue to obtain the vegetable oil characterized by:
a concentration of lauric acid which is at least 2 weight percent of the fatty acids of the vegetable oil;
a concentration of myristic acid which is at least 2 weight percent of the fatty acids of the vegetable oil;
a concentration of palmitic acid which is at least 20 weight percent of the fatty acids of the vegetable oil; and
a concentration of oleic acid which is no more than 35 weight percent of the fatty acids of the vegetable oil.

According to an aspect of some embodiments of the invention, there is provided a method of isolating dihydrouracil, the method comprising providing an oil according to any of the embodiments described herein and/or a composition comprising dihydrouracil according to any of the embodiments described herein, and isolating dihydrouracil from the oil and/or the composition.

According to some of any of the embodiments of the invention relating to a method of isolating dihydrouracil, providing an oil comprises preparing a vegetable oil according to the method of preparing a vegetable oil according to any of the embodiments described herein.

According to some of any of the embodiments of the invention relating to a method, the non-fruit tissue is an agricultural waste material.

According to some of any of the embodiments of the invention relating to a method, at least a portion of the non-fruit tissue is meristematic tissue.

According to some of any of the embodiments of the invention relating to a method, the method further comprises removal of non-meristematic tissue prior to the pressing.

According to some of any of the embodiments of the invention relating to a method, the pressing comprises cold pressing.

According to some of any of the embodiments of the invention relating to a method, the method further comprises separating a polysaccharide-containing fraction from an oil-containing fraction subsequently to the pressing.

According to some of any of the embodiments of the invention relating to separating a polysaccharide-containing fraction, separating a polysaccharide-containing fraction is effected by contacting a liquid obtained by pressing as described herein with a water-immiscible solvent.

According to some of any of the embodiments of the invention relating to a water-immiscible solvent, the water-immiscible solvent is dichloromethane.

According to some of any of the embodiments of the invention relating to an oil comprising esters of fatty acids, at least 20 weight percent of the fatty acids are palmitic acid.

According to some of any of the embodiments of the invention relating to an oil comprising esters of fatty acids, at least 50 weight percent of the fatty acids are selected from the group consisting of palmitic acid and linolelaidic acid.

According to some of any of the embodiments of the invention relating to an oil comprising esters of fatty acids, at least 5 weight percent of the fatty acids are stearic acid.

According to some of any of the embodiments of the invention relating to an oil comprising esters of fatty acids, at least 70 weight percent of the fatty acids are selected from the group consisting of palmitic acid, stearic acid and linolelaidic acid.

According to some of any of the embodiments of the invention relating to an oil comprising esters of fatty acids, no more than 10 weight percent of the fatty acids are oleic acid.

According to some of any of the embodiments of the invention relating to an oil comprising esters of fatty acids, no more than 10 weight percent of the fatty acids are selected from the group consisting of oleic acid, lauric acid and myristic acid.

According to some of any of the embodiments of the invention relating to an oil comprising esters of fatty acids, the esters of fatty acids comprise triglycerides.

According to some of any of the embodiments of the invention relating to an oil comprising esters of fatty acids, the oil further comprises at least one phytosterol.

According to some of any of the embodiments of the invention relating to an oil comprising at least one phytosterol, a total concentration of the at least one phytosterol is at least 20% of the oil by weight.

According to some of any of the embodiments of the invention relating to an oil comprising at least one phytosterol, a concentration of β-sitosterol is at least 15% of the oil by weight.

According to some of any of the embodiments of the invention relating to an oil comprising at least one phytosterol, a total concentration of esters of fatty acids and the at least one phytosterol is at least 90% of the oil by weight.

According to some of any of the embodiments of the invention relating to an oil, the oil further comprises dihydrouracil.

According to some of any of the embodiments of the invention relating to an oil, a concentration of dihydrouracil is at least 1% of the oil by weight.

According to some of any of the embodiments of the invention relating to an oil, the oil comprises esters of palmitic acid, linolelaidic acid, stearic acid, oleic acid, linoleic acid and pentadecanoic acid; β-sitosterol; stigmasterol; campesterol; cholesterol; cyclolanosterol; and dihydrouracil.

According to some of any of the embodiments of the invention relating to a method of obtaining oil by pressing date kernel tissue, the pressing is effected in a device with a rotating pole and an outlet configured for releasing the oil, wherein a diameter of said outlet is at least 5 mm.

According to some of any of the embodiments of the invention relating to a vegetable oil from date kernel tissue, the oil is obtained according to a method of obtaining vegetable oil from date kernel tissue according to any of the respective embodiments described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawing. With specific reference now to the drawing in detail, it is stressed that the particulars shown are by way of example and for purposes of illustrative discussion of embodiments of the invention. In this regard, the description taken with the drawing makes apparent to those skilled in the art how embodiments of the invention may be practiced.

In the drawing:

FIG. 1 presents a drawing depicting the anatomy of a date palm; of particular interest are the high and low offshoots, which may be utilized as raw material according to some embodiments of the invention (adapted from Zvi Bernstein [The Date Palm (Hebrew), Fruit Production and Marketing Council, Tel Aviv, 2004]).

### DESCRIPTION OF SPECIFIC EMBODIMENTS OF THE INVENTION

The present invention, in some embodiments thereof, relates to utilization of natural products, and more particularly, but not exclusively, to isolation of compositions from palm trees.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that considerable amounts of oil may be obtained from non-edible portions of the date palm, such as date palm offshoots and date kernels (which are generally regarded as agricultural waste materials in the date industry), with relatively high yields and low costs, using techniques suitable for industrial scales, such as cold pressing. The inventors have further uncovered additional compositions of potential economic value which may also be obtained from non-edible portions of the date palm.

While reducing the present invention to practice, the inventors have uncovered that the composition (e.g., fatty acid profile) of oil obtained from date palm offshoots by cold pressing is significantly different from that of other types of vegetable oil, including oil obtained from fruit tissue of various palm species, such as oil palm fruits or kernels, date kernels, and coconuts.

Method of Preparing Oil:

According to an aspect of some embodiments of the invention, there is provided a method of preparing a vegetable oil. The method, according to some embodiments described herein, comprises pressing a tissue of a date palm, for example, a non-fruit tissue and/or a date kernel tissue.

In some of any of the respective embodiments described herein, the method comprises pressing a non-fruit tissue of a date palm, for example, a non-fruit tissue of an offshoot of a date palm.

Herein, the terms “date palm” and “date tree” (which are used herein interchangeably) refer to any palm of the genus Phoenix, including domesticated and wild individuals, and including any cultivar thereof.

In exemplary embodiments, the date palm is Phoenix dactylifera.

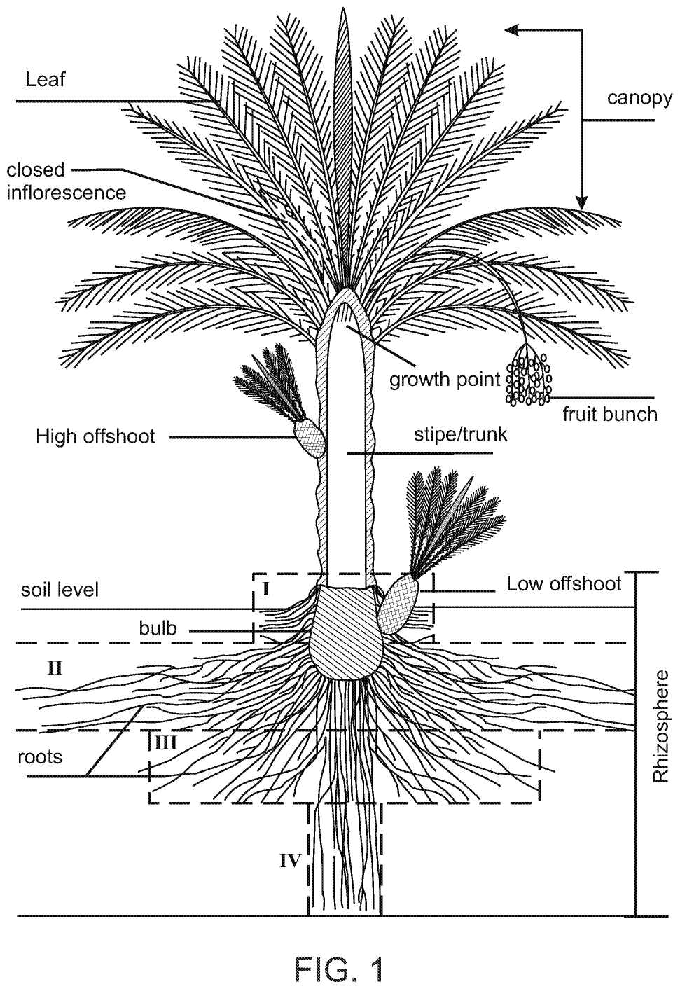

Herein, the term “date palm offshoot” refers to any trunk-like structure other than the central trunk of the date palm, with a growth point capable of growing leaves which is distinct from the growth point (and associated leaves) of the central trunk (see for example, FIG. 1). The offshoot may grow out of the central trunk (e.g., attached to the central trunk above ground; see for example, the “high offshoot” in FIG. 1) or the bulb or root system of the date palm (e.g., attached to the central trunk below ground; see for example, the “low offshoot” in FIG. 1). Offshoots attached to the central trunk below ground (e.g., via a common bulb or root system) may have the appearance of a separate tree (albeit one which tends to exit the ground somewhat diagonally), although they actually represents a portion of the same organism.

Herein, the term “oil” encompasses any hydrophobic substance.

Herein, the term “vegetable oil” refers to an oil (as defined herein) which is obtained from any portion(s) of a plant (e.g., a date palm), and is not intended to be further limiting.

Herein, the term “non-fruit tissue of a date palm” excludes tissue of a fruit of a date palm, including the pulp and the kernel (seed).

In addition, the non-fruit tissue and the kernel of a date palm are collectively referred to herein (interchangeably) as the “non-edible” portion of the date palm.

In some of any of the embodiments described herein, at least a portion of the non-fruit tissue is meristematic tissue. In some such embodiments, the method further comprises removal of at least a portion of non-meristematic tissue (e.g., parenchymatic tissue) prior to pressing, and optionally substantially all of the non-meristematic tissue (e.g., parenchymatic tissue).

Alternatively or additionally to obtaining oil from a non-fruit tissue of a date tree, the method according to some embodiments described herein comprises pressing date kernel tissue.

A notable advantage of the method according to any of the embodiments described herein is that it may utilize agricultural waste material (e.g., date palm offshoots and/or date kernels); and thus may complement, rather than compete with, the typical use of data palm for producing date fruits. For example, the removal of offshoots is a common agricultural practice, e.g., to enhance date fruit yield. In contrast, while a growth point of the central trunk may also be suitable for preparing oil according to the method described herein, it is expected that this would cause significant economic damage (e.g., resulting in possible death of the tree). Similarly, removal of date kernels is a common practice upon processing and packaging of dates and production of other date products.

Herein, the term “pressing” encompasses any application of pressure to a plant tissue which facilitates release of oil from the tissue; including e.g., squeezing, grinding, cutting, reaming, and centrifugation. In exemplary embodiments, pressing is effected by a rotating screw-like pole. Facilitating release of oil may optionally comprise breaking down the structure of the plant tissue (e.g., breaking cell walls to release cellular contents and/or the connections between cells), for example, by squeezing, grinding, reaming and/or cutting; and/or separating oil from plant solids (and optionally also aqueous liquids), for example, by forcing the oil (e.g., using one or more rotating screws, centrifugation, and/or hydraulic pressure) through a filter which blocks solids (e.g., fiber, screen and/or perforated barrier) and/or by separating oil from plant solids (and optionally also aqueous liquid) according to specific gravity (e.g., by centrifugation).

Pressing optionally comprises more than one stage; for example, a first stage for breaking down the plant tissue structure (e.g., to obtain a paste), and a second stage for separating oil from solids (e.g., the solids in an obtained paste) and optionally also from an aqueous liquid. Optionally, the plant tissue is churned or mixed (e.g., for at least 10 minutes and/or up to 3 hours) between the first stage and the second stage; for example, to promote aggregation of small oil droplets released in the first stage, which may facilitate oil separation in the second stage.

In some of any of the embodiments described herein, the pressing comprises cold pressing.

Herein, the term “cold pressing” refers to pressing wherein the plant tissue is at a temperature of less than 40° C. for the duration of the pressing process, and optionally at a temperature of less than 30° C., and optionally at a temperature of less than 27° C.

The method according to any of the embodiments described herein may optionally comprise crushing a plant tissue (e.g., a hard plant tissue, such as date kernel tissue) prior to pressing.

Pressing according to any of the embodiments described herein may optionally be effected using any technique and/or apparatus known in the art for obtaining oil from other plants (e.g., olive oil). Alternatively or additionally, a technique and/or apparatus known in the art for extracting juice (fruit juice) may be used, e.g., to obtain a liquid which comprises both oil and an aqueous phase, optionally followed by separation of the oil from the aqueous liquid (e.g., by decantation, centrifugation and/or using a water-immiscible solvent). Techniques and/or apparatuses suitable for obtaining oil from relatively soft tissue such as olives and/or for extracting juice from succulent tissue such as carrots may be particularly suitable for obtaining oil from relatively soft and/or succulent non-fruit tissue of date tree (according to any of the respective embodiments described herein); whereas techniques and/or apparatuses suitable for obtaining oil from relatively hard and/or dry tissue such as oil seeds (e.g., jojoba seeds) may be particularly suitable for obtaining oil from date kernel tissue (according to any of the respective embodiments described herein).

For example, pressing relatively soft tissue (e.g., non-seed tissue) according to any of the respective embodiments described herein may optionally be effected using an apparatus configured with relatively small openings (e.g., in a filter which blocks solids according to any of the respective embodiments described herein) for releasing fluid (e.g., oil), for example, less than 5 mm (e.g., no more than 4 mm) in diameter and/or less than 20 mm² (e.g., no more than 15 mm²) in area; whereas pressing relatively hard tissue (e.g., date kernel tissue) according to any of the respective embodiments described herein may optionally be effected using an apparatus configured with relatively large openings for releasing fluid (e.g., oil), for example, at least 5 mm (e.g., at least 6 mm or at least 7 mm) in diameter and/or at least 20 mm² (e.g., at least 30 mm² or at least 40 mm²) in area.

In some of any of the embodiments described herein, the method further comprises separating an aqueous and/or polysaccharide-containing fraction from an oil-containing fraction subsequently to pressing (e.g., when obtaining oil from a non-edible portion, such as non-fruit tissue, according to any of the respective embodiments described herein).

Separating an aqueous and/or polysaccharide-containing fraction may optionally be effected using centrifugation, for example, using techniques and/or apparatuses designed for separating aqueous fractions from other types of oil (e.g., olive oil).

Additionally or alternatively, separating an aqueous and/or polysaccharide-containing fraction is optionally effected by contacting a liquid obtained upon pressing with a water-immiscible solvent, optionally by forming a phase containing the water-immiscible solvent and oil-containing fraction which is readily separated from a more polar phase containing the aqueous and/or polysaccharide-containing fraction. The water-immiscible solvent may optionally then be evaporated to obtain the oil-containing fraction.

Dichloromethane is an exemplary water-immiscible solvent for separating an aqueous and/or polysaccharide-containing fraction from an oil-containing fraction. Hover the use other water-immiscible solvents is also contemplated; for example, nonpolar solvents such as alkanes and cycloalkanes (e.g., pentane, hexane, cyclopentane, cyclohexane), benzene and substituted derivatives thereof (e.g., toluene, xylene), chloroform, tetrachloromethane, and ethers (e.g., diethyl ether), and water-immiscible polar solvents such as certain esters (e.g., ethyl acetate) and alcohols (e.g., n-butanol, alcohols with 5 or more carbon atoms).

According to an aspect of some embodiments of the invention, there is provided a polysaccharide-containing fraction obtainable according to any of the embodiments of the method described herein which relate to separation of a polysaccharide-containing fraction (e.g., by pressing a non-edible portion, such as non-fruit tissue, of a date palm according to any of the respective embodiments described herein).

According to an aspect of some embodiments of the invention, there is provided a composition comprising a polar phase and a nonpolar phase, the composition being obtainable according to the method of obtaining a vegetable oil described herein (e.g., for obtaining oil from a non-edible portion, such as non-fruit tissue, of a date palm according to any of the respective embodiments), wherein the nonpolar phase comprises the vegetable oil (according to any of the embodiments described herein), and the polar phase comprises polysaccharides. The polar phase is optionally an aqueous and/or polysaccharide-containing fraction according to any of the respective embodiments described herein.

According to an aspect of some embodiments of the invention, there is provided a vegetable oil obtainable according to a method of obtaining a vegetable oil described herein, according to any of the respective embodiments. In some embodiments, the vegetable oil is obtainable by combining two or more distinct vegetable oils obtainable according to embodiments of a method described herein, e.g., combining oil obtainable from non-fruit tissue (according to any of the respective embodiments) and oil obtainable from date kernel tissue (according to any of the respective embodiments) to obtain a third oil with chemical characteristics intermediate between those of the oil obtainable from non-fruit tissue and the oil obtainable from date kernel tissue. In some embodiments, either the non-fruit tissue or the date kernels or both may optionally be waste material (e.g., agricultural waste material).

Oil:

According to an aspect of some embodiments of the invention, there is provided an oil comprising esters of fatty acids, for example, triglycerides (i.e., an ester of glycerol and three fatty acids). The oil is optionally obtained according to a method described herein of obtaining a vegetable oil from non-fruit tissue, according to any of the respective embodiments.

A wide variety of potential uses of an oil described herein (according to any of the respective embodiments) will be readily apparent to the skilled person, including, without limitation, uses as fuel, food and/or solvent.

Herein, the term “esters of fatty acids” refers to any compound comprising at least one ester bond formed from a fatty acid moiety and an alcohol moiety. For example, the alcohol moiety may optionally be a glycerol moiety, which may be attached to one, two or three fatty acid moieties, thus respectively forming a monoglyceride, a diglyceride, and a triglyceride.

Examples of monoglycerides and diglycerides include, without limitation, phosphoglycerides (wherein the glycerol is attached to a substituted or non-substituted phosphoryl group) such as phosphatidylcholine, phosphatidylethanolamine, phosphatidylinositol, phosphatidylserine and phosphatidic acid. Alternatively, the alcohol may be a simple alcohol (i.e., comprising only one —OH group) attached to a fatty acid—such esters are also known as “wax esters”.

Herein, the term “fatty acid” includes any compound comprising a chain of carbon atoms at least 4 carbon atoms in length which includes a —C(═O)OH group. Optionally, the carbon atom chain is at least 10 carbon atoms in length (e.g., from 10-30 carbon atoms in length). Optionally, the fatty acid has the formula R—C(═O)OH, wherein R is non-substituted aliphatic hydrocarbon chain which may be saturated or unsaturated.

In some of any of the embodiments described herein relating to esters of fatty acids, at least 50 weight percent of the esters of fatty acids are triglycerides. In some embodiments, at least 60 weight percent of the esters of fatty acids are triglycerides. In some embodiments, at least 70 weight percent of the esters of fatty acids are triglycerides. In some embodiments, at least 80 weight percent of the esters of fatty acids are triglycerides. In some embodiments, at least 90 weight percent of the esters of fatty acids are triglycerides. In some embodiments, at least 95 weight percent of the esters of fatty acids are triglycerides. In some embodiments, at least 98 weight percent of the esters of fatty acids are triglycerides. In some embodiments, at least 99 weight percent of the esters of fatty acids are triglycerides.

In some of any of the respective embodiments described herein, at least 10 weight percent of the fatty acids are linolelaidic acid; for example, wherein from 10 to 60 weight percent, or from 10 to 50 weight percent, or from 10 to 40 weight percent, or from 10 to 30 weight percent, or from 10 to 20 weight percent of the fatty acids are linolelaidic acid. In some such embodiments, at least 20 weight percent of the fatty acids are linolelaidic acid; for example, wherein from 20 to 60 weight percent, or from 20 to 50 weight percent, or from 20 to 40 weight percent, or from 20 to 30 weight percent of the fatty acids are linolelaidic acid. In some embodiments, at least 30 weight percent of the fatty acids are linolelaidic acid; for example, wherein from 30 to 60 weight percent, or from 30 to 50 weight percent, or from 30 to 40 weight percent of the fatty acids are linolelaidic acid.

As exemplified herein, linolelaidic acid is a fatty acid which is uncommon in nature, but is present in characteristically large proportions (largely in esterified form) in oil derived from non-fruit tissue of date palm, which is also rich in palmitic acid.

In some of any of the respective embodiments described herein, at least 20 weight percent of the fatty acids are palmitic acid; for example, wherein from 20 to 70 weight percent, or from 20 to 60 weight percent, or from 20 to 50 weight percent, or from 20 to 40 weight percent, or from 20 to 30 weight percent of the fatty acids are palmitic acid. In some such embodiments, at least 30 weight percent of the fatty acids are palmitic acid; for example, wherein from 30 to 70 weight percent, or from 30 to 60 weight percent, or from 30 to 50 weight percent, or from 30 to 40 weight percent of the fatty acids are palmitic acid. In some embodiments, at least 40 weight percent of the fatty acids are palmitic acid; for example, wherein from 40 to 70 weight percent, or from 40 to 60 weight percent, or from 40 to 50 weight percent of the fatty acids are palmitic acid.

In some of any of the respective embodiments described herein, the total of linolelaidic acid and palmitic acid is least 50 weight percent of the fatty acids; for example, wherein from 50 to 90 weight percent, or from 50 to 80 weight percent, or from 50 to 70 weight percent, or from 50 to 60 weight percent of the fatty acids are linolelaidic acid and/or palmitic acid. In some such embodiments, the total of linolelaidic acid and palmitic acid is least 60 weight percent of the fatty acids; for example, wherein from 60 to 90 weight percent, or from 60 to 80 weight percent, or from 60 to 70 weight percent of the fatty acids are linolelaidic acid and/or palmitic acid. In some embodiments, the total of linolelaidic acid and palmitic acid is least 70 weight percent of the fatty acids; for example, wherein from 70 to 90 weight percent, or from 70 to 80 weight percent of the fatty acids are linolelaidic acid and/or palmitic acid.

As further exemplified herein, the fatty acids of oil derived from non-fruit tissue of date palm may further comprise (in addition to linolelaidic acid and palmitic acid) substantial proportions of stearic acid and/or linoleic acid.

In some of any of the respective embodiments described herein, at least 5 weight percent of the fatty acids are stearic acid; for example, wherein from 5 to 20 weight percent, or from 5 to 15 weight percent, or from 5 to 10 weight percent of the fatty acids are stearic acid. In some such embodiments, at least 10 weight percent of the fatty acids are stearic acid; for example, wherein from 10 to 20 weight percent, or from 10 to 15 weight percent of the fatty acids are stearic acid.

In some of any of the respective embodiments described herein, the total of linolelaidic acid, palmitic acid and stearic acid is least 70 weight percent of the fatty acids; for example, wherein from 70 to 95 weight percent, or from 70 to 90 weight percent, or from 70 to 80 weight percent of the fatty acids are linolelaidic acid, palmitic acid and/or stearic acid. In some such embodiments, the total of linolelaidic acid, palmitic acid and stearic acid is least 80 weight percent of the fatty acids; for example, wherein from 80 to 95 weight percent, or from 80 to 90 weight percent of the fatty acids are linolelaidic acid, palmitic acid and/or stearic acid. In some embodiments, the total of linolelaidic acid, palmitic acid and stearic acid is least 85 weight percent of the fatty acids; for example, wherein from 85 to 95 weight percent, or from 85 to 90 weight percent of the fatty acids are linolelaidic acid, palmitic acid and/or stearic acid.

In some of any of the respective embodiments described herein, at least 0.2 weight percent of the fatty acids are pentadecanoic acid; for example, wherein from 0.2 to 5 weight percent, or from 0.2 to 2 weight percent, or from 0.2 to 1 weight percent, of the fatty acids are pentadecanoic acid. In some such embodiments, at least 0.5 weight percent of the fatty acids are pentadecanoic acid; for example, wherein from 0.5 to 5 weight percent, or from 0.5 to 2 weight percent, or about 1 weight percent of the fatty acids are pentadecanoic acid. Pentadecanoic acid is a relatively rare fatty acid, such that its presence in exemplary oil obtained as described herein in even small amounts may be distinctive.

As further exemplified herein, the fatty acids of oil derived from non-fruit tissue of date palm may be further characterized in having a low proportion of oleic acid, lauric acid and myristic acid. In contrast, oil derived from date kernels (fruit tissue) or oil palm fruit tends to be rich in oleic acid, whereas oil derived from coconut and oil palm kernels tends to be rich in lauric acid and myristic acid.

In some of any of the respective embodiments described herein, no more than 10 weight percent of the fatty acids in the oil are oleic acid. In some such embodiments, no more than 5 weight percent of the fatty acids are oleic acid. In some embodiments, no more than 2 weight percent of the fatty acids are oleic acid. In exemplary embodiments, no more than about 1 weight percent of the fatty acids is oleic acid.

In some of any of the respective embodiments described herein, no more than 5 weight percent of the fatty acids in the oil are lauric acid. In some such embodiments, no more than 2 weight percent of the fatty acids are lauric acid. In exemplary embodiments, less than 1 weight percent of the fatty acids is lauric acid.

In some of any of the respective embodiments described herein, no more than 5 weight percent of the fatty acids in the oil are myristic acid. In some such embodiments, no more than 2 weight percent of the fatty acids are myristic acid. In exemplary embodiments, less than 1 weight percent of the fatty acids is myristic acid.

In some of any of the respective embodiments described herein, the total of oleic acid, lauric acid and myristic acid is no more than 10 weight percent of the fatty acids. In some such embodiments, the total of oleic acid, lauric acid and myristic acid is no more than 5 weight percent of the fatty acids. In some such embodiments, the total of oleic acid, lauric acid and myristic acid is no more than 2 weight percent of the fatty acids. In some such embodiments, the total of oleic acid, lauric acid and myristic acid is no more than 1 weight percent of the fatty acids.

An oil obtained from date kernel tissue according to any of the respective embodiments described herein is optionally characterized by the presence of relatively short fatty acids such as lauric acid and/or myristic acid. In some embodiments, the fatty acids of the oil comprise at least 5 weight percent lauric acid, optionally at least 10 weight percent. In some embodiments, the fatty acids of the oil comprise at least 5 weight percent myristic acid, optionally at least 10 weight percent. In some embodiments, the total concentration of lauric acid and myristic acid is at least 8 weight percent of the fatty acids, optionally at least 16 weight percent.

Alternatively or additionally, an oil obtained from date kernel tissue according to any of the respective embodiments described herein is optionally characterized by a relatively high proportion of oleic acid (e.g., at least 40 weight percent of the fatty acids) and/or by a relatively low proportion of palmitic acid (e.g., no more than 30 weight percent or no more than 25 weight percent of the fatty acids).

As exemplified herein, different oils obtained from various non-edible portions of the date palm, such as an oil obtained from date kernel tissue (e.g., according to any of the respective embodiments described herein) and an oil obtained from non-fruit tissue of date palm (e.g., according to any of the respective embodiments described herein), may have significantly different compositions (e.g., with respect to fatty acid composition thereof), and may complement each other by being combined to include a desirable composition of fatty components. Such a combination may optionally be used as an alternative to currently available oil compositions that are produced, for example, from oil palm fruit (a source of palmitic acid), oil palm kernel and/or coconut (sources of shorter fatty acids) and combinations thereof.

An oil obtained by combining oil from date kernel tissue and oil from non-fruit tissue according to any of the respective embodiments described herein is optionally characterized by the presence (e.g., due to the oil from date kernel tissue) of relatively short fatty acids such as lauric acid and/or myristic acid. In some embodiments, the fatty acids of the oil comprise at least 2 weight percent lauric acid, optionally at least 5 weight percent, and optionally at least 8 weight percent. In some embodiments, the fatty acids of the oil comprise at least 2 weight percent myristic acid, optionally at least 5 weight percent, and optionally at least 8 weight percent. In some embodiments, the total concentration of lauric acid and myristic acid is at least 4 weight percent of the fatty acids, optionally at least 8 weight percent, and optionally at least 12 weight percent.

Alternatively or additionally, an oil obtained by combining oil from date kernel tissue and oil from non-fruit tissue according to any of the respective embodiments described herein is optionally characterized (e.g., due to the oil from non-fruit tissue) by a proportion of oleic acid which is not especially high (e.g., no more than 35 weight percent or no more than 30 weight percent or no more than 25 weight percent of the fatty acids) but also (e.g., due to the oil from date kernel tissue) not especially low (e.g., at least 5 weight percent or at least 10 weight percent or at least 20 weight percent of the fatty acids).

Alternatively or additionally, an oil obtained by combining oil from date kernel tissue and oil from non-fruit tissue according to any of the respective embodiments described herein is optionally characterized (e.g., due to the oil from date kernel tissue) by a proportion of palmitic acid which is not especially high (e.g., no more than 40 weight percent or no more than 35 weight percent or no more than 30 weight percent of the fatty acids) but also (e.g., due to the oil from non-fruit tissue) not especially low (e.g., at least 20 weight percent or at least 25 weight percent or at least 30 weight percent of the fatty acids).

In some of any of the respective embodiments described herein, the oil (whether derived from date kernel tissue or from non-fruit tissue) further comprises at least one phytosterol.

Herein, the term “phytosterol” refers to any compound which naturally occurs in at least one plant, and which comprises a gonane (perhydrocyclopenta[a]phenanthrene) skeleton (which may optionally be saturated or unsaturated) substituted by at least one hydroxy group and optionally by one or more additional substituents, for example, alkyl and/or alkenyl, and two substituents which together form a cycloalkyl ring (e.g., a 3-membered ring).

Examples of phytosterols include, without limitation, β-sitosterol, γ-sitosterol, stigmasterol, campesterol, cholesterol, cyclolanosterol, avenasterol, brassicasterol, fucosterol, isofucosterol, Δ7-stigmasterol, stigmastanol, campestanol, α-ergostenol, cyclolanostenol, and cycloartenol.

In some of any of the embodiments described herein relating to an oil comprising at least one phytosterol, the phytosterol comprises, at least one of, or at least two of, or at least three of, or at least four of, or all five of β-sitosterol, stigmasterol, campesterol, cholesterol, and cyclolanosterol (optionally at a concentration of at least 1% by weight of the oil). In some such embodiments, a concentration of at least one of, or at least two of, or at least three of, or at least four of, or all five of the aforementioned sterols in the oil is at least 1 weight percent, or at least 2 weight percent, or at least 3 weight percent, or at least 5 weight percent.

In some of any of the embodiments described herein relating to an oil comprising at least one phytosterol, the two most abundant phytosterols, or the three most abundant phytosterols, or the four most abundant phytosterols, or the five most abundant phytosterols in the oil are selected from β-sitosterol, stigmasterol, campesterol, cholesterol, cyclolanostenol and cyclolanosterol.

In some of any of the embodiments described herein relating to an oil comprising at least one phytosterol, β-sitosterol and stigmasterol are among the five most abundant phytosterols, or among the four most abundant sterols or are the three most abundant phytosterols in the oil.

In some of any of the embodiments described herein relating to an oil comprising at least one phytosterol, at least 30 weight percent, or at least 40 weight percent, or at least 50 weight percent, or at least 60 weight percent, or at least 70 weight percent of the total amount of phytosterol in the oil is β-sitosterol.

In some of any of the embodiments described herein relating to an oil, a concentration of β-sitosterol in the oil is at least 15% by weight; for example, from 15 to 50%, or from 15 to 40%, or from 15 to 30% by weight. In some such embodiments, the concentration of β-sitosterol in the oil is at least 20% by weight; for example, from 20 to 50%, or from 20 to 40%, or from 20 to 30% by weight. In some such embodiments, the concentration of β-sitosterol in the oil is at least 25% by weight; for example, from 25 to 50%, or from 25 to 40%, or from 25 to 35% by weight.

In some of any of the respective embodiments, a total concentration of the at least one phytosterol is at least 20% of the oil by weight; for example, from 20 to 60%, or from 20 to 50%, or from 20 to 40%, or from 20 to 30% by weight. In some such embodiments, a total concentration of the at least one phytosterol is at least 30% of the oil by weight; for example, from 30 to 60%, or from 30 to 50%, or from 30 to 40%, or from 20 to 30% by weight. In some of any of the aforementioned embodiments, at least 30 weight percent, or at least 40 weight percent, or at least 50 weight percent, or at least 60 weight percent, or at least 70 weight percent of the total amount of phytosterol in the oil is β-sitosterol.

In some of any of the respective embodiments, a total concentration of the phytosterol(s) in the oil (according to any of the respective embodiments described herein) and the esters of fatty acids in the oil (according to any of the respective embodiments described herein) is at least 90% of the oil by weight; for example, in a range of from 90 to 99%, or from 90 to 97% by weight. In some such embodiments, at least 30 weight percent, or at least 40 weight percent, or at least 50 weight percent, or at least 60 weight percent, or at least 70 weight percent of the total amount of phytosterol in the oil is β-sitosterol; and/or at least 50 weight percent, or at least 60 weight percent, or at least 70 weight percent, or at least 80 weight percent, or at least 90 weight percent of the total amount of esters of fatty acids are triglycerides (according to any of the respective embodiments described herein).

In some of any of the embodiments described herein relating to an oil (e.g., an oil obtained from a non-edible portion of a date palm, such as non-fruit tissue, according to any of the respective embodiments described herein), the oil further comprises dihydrouracil, for example at a concentration of at least 1% (e.g., from 1% to 10%, or from 1% to 5%) of the oil by weight, or at least 2% (e.g., from 2% to 10%, or from 2% to 4%) of the oil by weight, or at least 3% (e.g., from 3% to 10%, or from 3% to 5%) of the oil by weight.

In some of any of the embodiments described herein relating to an oil (e.g., an oil obtained from a non-edible portion of a date palm, such as non-fruit tissue, according to any of the respective embodiments described herein), the oil comprises esters of palmitic acid, linolelaidic acid, stearic acid, oleic acid, linoleic acid and pentadecanoic acid; β-sitosterol; stigmasterol; campesterol; cholesterol; cyclolanosterol; and dihydrouracil. In some such embodiments, the concentrations of any one of more of the aforementioned compounds is according to any of the respective embodiments described herein.

An oil according to any of the embodiments described herein may be used in any of a variety of applications for which vegetable oil is suitable. Examples of such uses include, without limitation, in food and/or cooking (e.g., for flavor, as shortening, to reduce sticking of ingredients, and/or as cooking oil); as electrical insulators; as hydraulic fluid; for lubrication; as carriers for lipophilic ingredients (e.g., in medicaments and cosmetic products); for manufacture of detergents and/or wax (e.g., in soaps, cosmetic products and/or candles); and/or fuel, e.g., for manufacture of biodiesel. Manufacture of various fatty acid esters may optionally be accomplished by transesterification with an alcohol, e.g., with methanol to prepare a methyl fatty acid ester (e.g. for use as biodiesel), or with higher alcohols to form a wax. Additionally or alternatively, fatty acids (and salts thereof) may be generated from the oil by hydrolysis (e.g., saponification), and these may be used per se (e.g., as a detergent) and/or reacted further, e.g., esterified with an alcohol to form an ester.

Additional Obtainable Compositions:

As exemplified herein, additional compositions may optionally be obtained from date non-fruit tissue such as agricultural waste material (e.g., a non-fruit tissue of date palm according to any of the respective embodiments described herein), for example, to offset at least some of the costs of producing dates and/or a date palm-derived vegetable oil described herein.

Such compositions may optionally be used as animal feed, biofuel, and/or as a raw material for isolating potentially valuable organic compounds (e.g., as described herein).

In some of any of the embodiments described herein relating to such compositions, the composition is obtained by solvent extraction of the non-fruit tissue (e.g., as opposed to pressing as described herein) using a suitable solvent. Solvent extraction comprises contacting non-fruit tissue (preferably crushed and/or ground tissue) with a solvent (which may comprise a single compound or a mixture of compounds), e.g., for at least 1 hour, or for at least 3 hours or for at least 6 hours or for at least 12 hours or for at least 24 hours. The solvent may optionally then be evaporated, for example, using reduced pressure without substantial heating (e.g., using a rotary evaporator) in order to avoid heat-induced damage to extracted compounds.

The non-fruit tissue subjected to extraction may optionally be tissue discarded from a method of obtaining oil according to any of the respective embodiments described herein; for example, parenchymatic tissue (e.g., when the oil is obtained by pressing meristematic tissue) and/or a solid residue upon removal of oil.

In some of any of the respective embodiments, the extraction is effected using a solvent which is a polar solvent. In exemplary embodiments, the polar solvent comprises water. In some embodiments, a nonpolar organic solvent (e.g., hexane) is also used during extraction, thus reducing an amount of nonpolar compounds in the obtained extract.

The skilled person will be readily capable of determining whether a given solvent is polar or nonpolar.

The compositions according to any of the respective embodiments described herein may optionally be in a fluid (e.g., aqueous) form, such as a solution, suspension, slurry and/or gel, or in a solid form (e.g., a substantially dry solid).

As exemplified herein, extraction may also be effected using a mixture of dichloromethane and methanol as solvent.

Alternatively, extraction may optionally be effected (according to any of the respective embodiments described herein) using a solvent which comprises any mixture (e.g., solution) of a nonpolar solvent and a polar solvent (according to any of the respective embodiments described herein).

According to an aspect of some embodiments of the invention, there is provided a composition comprising at least 5, or at least 10, or at least 14, or all 15 of the following compounds: asparagine, isoleucine, threonine, pipecolic acid, 5-hydroxypipecolic acid, allose, arabinose, galactose, ribose, talose, myo-inositol, linolenic acid, stearic acid, stearyl alcohol, and dihydrouracil. In some such embodiments, a concentration of each of at least 5, or at least 10, or at least 14, or all 15 of the aforementioned compounds is at least 1% by dry weight of the composition, or at least 2%, or at least 3%, or at least 4% or at least 5%, by dry weight of the composition.

As exemplified herein, such a composition may optionally be obtained by extraction of meristematic non-fruit tissue of date palm (according to any of the respective embodiments described herein) using a polar solvent, optionally water.

In some of any of the respective embodiments, the composition further comprises at least 3, or at least 6, or at least 9, or all 10 of the following compounds: alanine, aspartate, glutamate, proline, serine, valine, N-methyl-leucine, xylose, glycerol, and palmitic acid. In some such embodiments, a concentration of each of at least five, or at least 10, or at least 14, or all 10 of the aforementioned compounds is at least 0.1% by dry weight of the composition, or at least 0.2%, or at least 0.5%, or at least 1%, or at least 2%, by dry weight of the composition.

According to an aspect of some embodiments of the invention, there is provided a composition comprising at least five, or at least 10, or at least 15, or all 16 of the following compounds: 5-hydroxypipecolic acid, hydroxyisocaproic acid, fructose, galactose, lyxose, psicose, rhamnose, ribose, sorbose, talose, trehalose, xylose, arabinoic acid, malic acid, citric acid, and kaur-16-ene. In some such embodiments, a concentration of each of at least five, or at least 10, or at least 15, or all 16 of the aforementioned compounds is at least 1% by dry weight of the composition, or at least 2%, or at least 3%, or at least 4% or at least 5%, by dry weight of the composition.

As exemplified herein, such a composition may optionally be obtained by extraction of parenchymatic non-fruit tissue of date palm (according to any of the respective embodiments described herein) using a mixed polarity solvent, optionally a mixture of dichloromethane and methanol (e.g., at a 2:1 ratio of dichloromethane to methanol).

In some of any of the respective embodiments, the composition further comprises at least 3, or at least 6, or all 7 of the following compounds: norvaline, arabinose, glucose, maltose, mannose, sucrose, and palmitic acid. In some such embodiments, a concentration of each of at least 3, or at least 6, or all 7 of the aforementioned compounds is at least 0.1% by dry weight of the composition, or at least 0.2%, or at least 0.5%, or at least 1%, or at least 2%, by dry weight of the composition.

In some of any of the respective embodiments, the composition further comprises esters of fatty acids (as defined herein, according to any of the respective embodiments), wherein at least 80 weight percent (and optionally at least 90 or 95 weight percent) of the fatty acids are palmitic acid. Optionally the fatty acids further include linolenic acid and stearic acid; for example, wherein at least 1 weight percent, or at least 2 or at least 3 or at least 4 weight percent of the fatty acids are linolenic acid and/or stearic acid. As exemplified herein, such a composition may optionally be obtained by extraction of a combination of meristematic and parenchymatic non-fruit tissue using a mixed polarity solvent (according to any of the respective embodiments described herein), e.g., wherein the meristematic tissue serves as a primary source of the aforementioned fatty acid esters.

Potentially valuable compounds which may optionally be extracted from one or more of the compositions described hereinabove include, without limitation, hydroxypipecolic acid, which may inhibit blood platelet aggregation; kaur-16-ene, which may exhibit anti-inflammatory, antifungal and/or analgesic activity; norvaline, which may promote tissue generation and/or muscle growth (e.g., for use in a bodybuilding supplement) and/or inhibit arginase (e.g., for use in treating Alzheimer's disease); and/or dihydrouracil, which may be used as an antiviral compound.

Additionally or alternatively, one or more amino acids, saccharides and/or organic acids obtainable one or more of the compositions described hereinabove may optionally be used as food additives or nutritional supplements, for example, as a flavorant, as a nutrient (e.g., amino acid(s)), for caloric value or for texture (e.g., amino acid(s) and/or saccharide(s)), and/or to regulate acidity (e.g., organic acid(s)).

According to an aspect of some embodiments of the invention, there is provided a method of isolating dihydrouracil, the method comprising providing an oil and/or composition described herein (according to any of the embodiments described herein relating to an oil and/or composition which comprises uracil), and isolating dihydrouracil from the oil and/or composition (e.g., using procedures known in the art). In some such embodiments, the oil is obtained by a method of preparing a vegetable oil, according to any of the respective embodiments described herein.

It is expected that during the life of a patent maturing from this application many relevant techniques and apparatuses useful in pressing and extraction of plant tissue will be developed and the scope of the terms “pressing”, “extract”, “extraction” and grammatical variants thereof are intended to include all such new technologies a priori.

Optionally, a polar solvent described herein (according to any of the respective embodiments) is a protic solvent, that is, compound capable of readily donating a proton (H⁺) to solutes (e.g., a compound comprising a hydroxy group). In any of embodiments in which a nonpolar solvent is used in combination with a polar solvent, the nonpolar solvent is optionally aprotic (i.e., not protic, as this term is described herein).

Optionally, a polar solvent described herein (according to any of the respective embodiments) has a dielectric constant of at least 5 at a temperature of 25° C. In any of the embodiments described herein in which a nonpolar solvent is used in combination with a polar solvent, the nonpolar solvent optionally has a dielectric constant of less than 5 at a temperature of 25° C.

Optionally, a polar solvent described herein (according to any of the respective embodiments) is water or a water-miscible solvent. In any of the embodiments described herein in which a nonpolar solvent is used in combination with a polar solvent, the nonpolar solvent is optionally water-immiscible.

Herein, the term “water-miscible” refers to a compound capable of mixing with water in all possible proportions to form a homogeneous solution (at a temperature of 25° C.); and “water-immiscible” refers to a compound which is incapable of mixing with water (at a temperature of 25° C.) to form a homogeneous solution in at least some proportions (of the compound and water).

As used hereinabove, the term “about” refers to ±20%, and in optional embodiments to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### EXAMPLE 1

### Oil Obtained from Date Tree Offshoots by Organic Solvent Extraction or Cold Press Extraction

Materials and Methods

Organic Solvent Extraction:

To obtain date palm (P. dactylifera) oil from the offshoots sampled, the leaves were peeled off and the offshoot was cut in half to reveal its inner part with a chain saw. For solvent extraction, both the meristematic zone (about 10-30% of the offshoot total volume) and the connective parenchymatic (about 70-90% of the offshoot total volume) tissue were sampled separately. 150 grams (fresh weight) of each area was cut out with a knife and crushed manually, then ground very slowly using short pulses of an electric grinder.

For mixed-polarity extraction, 400 ml of a mixture of dichloromethane and methanol (2:1, v/v) was added to the samples, followed by stirring for 40 minutes. The supernatant was decanted to a clean vial. The obtained extract was dried using a gentle stream of nitrogen, with no heating of the extract.

Other samples were extracted with a mixture of n-hexane and water (1:1, v/v) added to ground plant tissue and separated to its polar and nonpolar phases using a separation bottle followed by a Buchner filtration apparatus.

The minimized aliquot of the extracts (about 1 ml each) were transferred to a 2 ml sterile glass vials and 100 μl of a known amount of 1-nonadecanol (C₁₉-alcohol) was added to the concentrated extracts in order to serve as an internal standard for yield assessment. The remaining solvent was dried under a gentle stream of nitrogen until complete dryness.

Cold Press Extraction:

The offshoots of date palm (P. dactylifera) were cut in half with a chain saw to reveal their inner part. The internal growth bud was entirely removed. The plant tissue (approximately 1 kg) was crushed and the (primarily aqueous) juice was squeezed out using a semi-industrial hard fruit juice maker (Hurom). Alternatively, this step was performed using a device comprising a rotating screw-like pole (no heat or prior crushing) and a nozzle diameter of 4 mm. The obtained clear juice was centrifuged at 4000 r.p.m for 12 minutes, and then the supernatant was decanted into a separation funnel. 100 ml of dichloromethane was added and then shaken well, in order to separate the sugars from the oil. After separation, the lower, clear odorless fraction contained purified oil with a light yellow tone, in dichloromethane (which was evaporated completely prior to analysis). The upper fraction, having a clear honey-brown color, contained polysaccharides.

Analysis of Contents:

Prior to analysis, 100 μl of N,O-bis(trimethylsilyl)trifluoroacetamide (BSTFA) containing 1% trimethylchlorosilane (TMCS) were added to the samples, followed by heating at 70° C. for 20 minutes, which promoted degradation of triglycerides in the oil to free fatty acids and monoglycerides. Silylation (with trimethylsilyl groups) renders many compounds more volatile, thereby facilitating analysis by gas chromatography. From each sample, 1 μl was injected into a gas chromatograph (GC) with mass selective detector (MS), using a 1:10 split ratio injection mode. Gas chromatography/mass spectrometry (GC/MS) analyses were carried out using an HP 7890 gas chromatograph coupled to an HP 5973 mass spectrometer (electron multiplier potential 2 kV, filament current 0.35 mA, electron energy 70 eV, spectra recorded over the range m/z 40-800). An Agilent™ 7683 autosampler was used for sample introduction. Helium was used as a carrier gas at a constant flow of 1.1 ml/second. An isothermal hold at 50° C. was kept for 2 minutes, followed by a heating gradient of 10° C. per minute to 325° C., with the final temperature held for 10 minutes. An HP-5 ms 5% cross-linked phenylmethyl siloxane capillary column (30 m, 0.25 mm internal diameter, with a 0.25 μm film thickness) was used for separation and the injection port temperature was 280° C. The MS interface temperature was 300° C. Peak assignments were carried out with the aid of library spectra (NIST 1.6) and compared with published data and MS data obtained from the injection of standards obtained from Sigma-Aldrich.

Results

Organic Solvent Extraction:

An organic solvent extract of different parts of date palm heart was prepared and separated into polar and nonpolar fractions, according to procedures described hereinabove. The contents of the obtained extract and fractions thereof were determined by gas chromatography/mass spectrometry (according to procedures described hereinabove), as summarized in Table 1 below. 7 ml of dry oil was obtained from 150 grams meristematic tissue, which represents a yield of almost 50 ml per kg of raw material.

As shown in Table 1 below, the polar phase of the extract obtained from date palm mostly comprised amino acids (such as alanine, valine, proline, isoleucine, N-methyl-leucine, aspartic acid and asparagine), monosaccharides (such as galactose, arabinose, myo-inositol and mannose), and some minute amounts of components such as acid derivatives of monosaccharides (such as L-manno-heptonic acid, glucuronic acid and mannoic acid; not shown).

The oil extracted from the meristematic zone was composed almost exclusively of palmitic acid (C_{16:0}).

As further shown in Table 1, the extract obtained from the connective parenchymatic tissue comprised mainly monosaccharides (such as fructose, galactose, arabinose, xylose, glucose and mannose) and their acid derivatives (such as arabinoic acid), along with disaccharides (such as sucrose and trehalose).

TABLE 1
Chemical profile of various fractions extracted from meristematic
and parenchymatic regions of date palm heart tissues

| | |
|---|---|
| Compound | Relative amount (%) |

A. meristematic zone - water extraction

| | |
|---|---|
| Standard amino acids | About 42 |
| L-asparagine | 15.48 |
| L-isoleucine | 11.61 |
| L-threonine | 4.80 |
| L-aspartic acid | 1.97 |
| L-proline | 1.74 |
| L-glutamic acid | 1.72 |
| L-serine | 1.63 |
| L-alanine | 1.59 |
| L-valine | 1.34 |
| Non-standard amino acids | About 12 |
| 5-hydroxypipecolic acid * | 6.10 |
| pipecolic acid | 3.97 |
| N-methyl-leucine | 1.47 |
| Saccharides | About 39 |
| D-allose | 11.61 |
| arabinose | 7.86 |
| D-galactose | 6.54 |
| D-talose | 4.07 |
| myo-inositol | 2.88 |
| D-ribose | 2.81 |
| glycerol | 1.06 |
| D-xylose | 0.99 |
| stearic acid (C_{18:0}) | 3.96 |
| dihydrouracil | 2.89 |
| octadecanol (stearyl alcohol) | 2.11 |
| linolenic acid (C_{18:2}) | 2.11 |
| palmitic acid (C_{16:0}) | 1.73 |

B. meristematic tissue - mixed polarity extraction (oil)

| | |
|---|---|
| palmitic acid (C_{16:0}) | 90.09 |
| linolenic acid (C_{18:2}) | 4.02 |
| stearic acid (C_{18:0}) | 4.89 |

C. parenchymatic tissue - mixed polarity extraction

| | |
|---|---|
| Mono- and di-saccharides | About 61 |
| D-ribose | 11.55 |
| lyxose | 7.73 |
| talose | 5.47 |
| galactose | 5.46 |
| D-xylose | 4.18 |
| L-rhamnose | 3.98 |
| trehalose | 3.71 |
| L-sorbose | 3.11 |
| arabinoic acid | 3.03 |
| D-psicose | 2.91 |
| fructose | 2.29 |
| arabinose | 1.87 |
| maltose | 1.81 |
| mannose | 1.68 |
| glucose | 1.58 |
| sucrose | 0.21 |
| citric acid | 9.15 |
| kaur-16-ene **** | 6.96 |
| 5-hydroxypipecolic acid * | 6.28 |
| hydroxyisocaproic acid | 3.02 |
| L-malic acid ** | 2.36 |
| L-norvaline *** | 1.51 |
| palmitic acid (C_{16:0}) | 0.63 |

* Inhibits blood platelets aggregation [El Khalabi et al., J Prep Biochem Biotechnol 2000, 30: 295-304; Panda, H. Handbook on Herbal Drugs and its Plant Sources: National Institute of Industrial Research, Delhi, India, 2002; p 512]
** Food additive (e.g., acidity regulator)
*** Promotes tissue generation and muscle growth [De Santo et al., PNAS 2005, 102: 4185-4190], arginase inhibitor [Porporatto et al., Biochem Biophys Res Commun 2003, 304: 266-272]
**** Exhibits anti-inflammatory, antifungal and analgesic activity [Cheng et al., J Agric Food Chem 2005, 53: 614-619; Karioti et al., J Agric Food Chem 2004, 52: 8094-8098]

Cold Press Extraction:

In an alternative process, an oil was obtained from the growth points of date palm by cold pressing, according to procedures described hereinabove (as opposed to solvent extraction described hereinabove). 600 ml of oil was obtained per 1 kg plant material using this technique.

The contents of the obtained oil were determined by gas chromatography/mass spectrometry (according to procedures described hereinabove), as summarized in Tables 2 and 3 below.

TABLE 2
Composition of cold-pressed oil obtained from date palm (as determined
after derivatization with BSTFA (N,O-bis(trimethylsilyl)fluoroacetamide)
containing 1% of trimethylchlorosilane)

| | | | Relative amount |
|---|---|---|---|
| | Compound | | (by mass) |
| dihydrouracil | | | 3% |
| | fatty acids | C_{15:0} | 0.5% |
| | | C_{16:0} | 16.7% |
| | | linolelaidic | 14.1% |
| | | acid | |
| | | C_{18:2} | 4.5% |
| | | C_{18:1} | 0.5% |
| | | C_{18:0} | 4.8% |
| | monoacyl- | linolelaidic | 2.4% |
| | glycerides | acid | |
| | (MAG) | C_{18:2} | 4.7% |
| | | C_{18:1} | 5.2% |
| | | C_{18:0} | 5.7% |
| | sterols | cholesterol | 1% |
| | | campesterol | 2.5% |
| | | stigmasterol | 4.3% |
| | | β-sitosterol | 29.2% |
| | | cyclolanosterol | 0.9% |

As shown in Table 2, the oil obtained by cold pressing was rich in sterols (primarily β-sitosterol); and a significant amount (3%) of dihydrouracil (an antiviral compound) was also present.

TABLE 3
Fatty acid profile of cold-pressed oil obtained from date palm

| | Percentage of |
|---|---|
| Fatty acid | total fatty acids |
| C_{15:0} | 1% |
| C_{16:0} | 40.6% |
| linolelaidic | 34.4% |
| acid | |
| C_{18:2} | 10.9% |
| C_{18:1} | 1.2% |
| C_{18:0} | 11.7% |

As shown in Tables 2 and 3, the fatty acids of the oil obtained by cold pressing primarily composed of palmitic acid and linolelaidic acid (an omega-6 trans-fatty acid). As further shown therein, the fatty acids included little or no oleic acid (C_{18:1}), myristic acid (C_{14:0}) or lauric acid (C_{12:0}).

It is notable that oleic acid is an abundant component of a wide variety of vegetable oils, whereas lauric and myristic acid are characteristic of various oils obtained from the fruit of palm trees, as summarized in Table 4. As further shown in Table 4, the oil obtained by cold pressing was considerably different from the oil obtained by organic solvent extraction.

TABLE 4
Comparison of fatty acid profile of palm tree oil prepared as described herein with other
oils obtained from palm species

| | Percentage of total fatty acids | | | | | | | | | |
|---|---|---|---|---|---|---|---|---|---|---|
| | | | | | | | | | | Linolelaidic |
| Oil source | C_{8:0} | C_{10:0} | C_{12:0} | C_{14:0} | C_{15:0} | C_{16:0} | C_{18:0} | C_{18:1} | C_{18:2} | acid |
| Date palm tree (P. | — | — | — | — | 1 | 41 | 12 | 1 | 11 | 34 |
| dactylifera) | | | | | | | | | | |
| cold-pressed | | | | | | | | | | |
| Date palm tree (P. | — | — | — | — | — | 91 | 4 | — | 4 | — |
| dactylifera) | | | | | | | | | | |
| organic solvent | | | | | | | | | | |
| extraction | | | | | | | | | | |
| Oil palm fruit | — | — | — | 1 | — | 43 | 4 | 42 | 10 | — |
| (E. oleifera)* | | | | | | | | | | |
| Date kernel | — | 1 | 10 | 7.5 | — | 10 | 1.5 | 50 | 19 | — |
| (P. dactylifera)** | | | | | | | | | | |
| Oil palm kernel | 4 | 3 | 49 | 16 | — | 8 | 2 | 15 | 3 | — |
| (E. oleifera)* | | | | | | | | | | |
| Coconut (C. | 7 | 5 | 49 | 20 | — | 9 | 3 | 6 | 1 | — |
| nucifera)*** | | | | | | | | | | |

*Coursey, D. G., et al. [Oil Palm News 1984, 28:11-17]; and Tugendhaft, Y. [Morphological, Biochemical and Genetic Characterization of Landrace Olive Trees in Israel, PhD Thesis, Hebrew University, Jerusalem, 2015]
**Pearson, D. [Chemical Analysis of Foods, 7th Ed. London: Churchill, Livingstone, 1976]
***Internal analysis (data not shown) of coconut oil (obtained from Ness Oils Ltd).

These results indicate that oil can be obtained from date palm (non-fruit) tissue by cold pressing, and separated from aqueous content, in a manner similar to preparation of olive oil. The oil is characterized by a high concentration of sterols and by an unusual free fatty acid profile, particularly the presence of considerable amounts of the uncommon linolelaidic acid, and also contains significant amounts of dihydrouracil, a potentially valuable compound. Furthermore, a polysaccharide fraction is also obtained.

The obtained yield of 600 ml per kg plant material suggests that a date palm may provide an annual yield of close to 4 liters per tree (comparable to olive trees), considering that an offshoot may provide about 1.5-2 kg of raw material, and each tree may grow up to 4 offshoots per year on average.

### EXAMPLE 2

### Oil Obtained from Date Kernels by Cold Press Extraction

Date kernels, a waste product of the date tree industry, were washed under tap water for 10 seconds and then dried for 12 hours. The kernels were then subjected to cold pressing as follows: the date kernels were subjected to pressing using a commercial cold press mill with a rotating screw-like pole which was constantly heated by hot air blower directed towards the pole, and a nozzle diameter of 7 mm. The yield of oil was about 100-250 μl oil per gram of date kernels.

The composition of the obtained oil was determined using gas chromatography/mass spectrometry, using procedures similar to those described in Example 1. 10 μl oil was added to 90 μl BSTFA with 1% TMCS, and the resulting silylated samples were analyzed by gas chromatography/mass spectrometry. The contents of the obtained oil are summarized in Tables 5 and 6 below.

TABLE 5
Composition of cold-pressed date kernel oil (as determined
after derivatization with BSTFA (N,O-bis(trimethylsilyl)fluoroacetamide)
containing 1% of TMC (trimethylchlorosilane)

| | | | Relative |
|---|---|---|---|
| | | | amount |
| | Compound | | (by mass) |
| | fatty acids | C_{12:0} | 1.5% |
| | | C_{14:0} | 1.1% |
| | | C_{16:0} | 4.0% |
| | | C_{18:2} | 1.8% |
| | | C_{18:1} | 8.3% |
| | | C_{18:0} | 1.5% |
| | monoacyl- | linolelaidic acid | 2.4% |
| | glycerides | C_{18:2} | 4.7% |
| | (MAG) | C_{18:1} | 5.2% |
| | | C_{18:0} | 5.7% |
| Monoacyl-(C_{16:0})-glyceride (MAG) | | | 1.3% |
| squalene | | | 1.1% |
| (E)-bicycloeicosane | | | 3.7% |
| | sterols | campesterol | 5.2% |
| | | stigmasterol | 4.7% |
| | | γ-sitosterol | 2.3% |
| | | β-sitosterol | 35.4% |
| | | isofucosterol | 4.2% |
| | | cyclolanostenol | 19.0% |
| | | cyclolanosterol | 5.6% |
| | | α-ergostenol | 2.4% |

TABLE 6
Fatty acid profile of cold-pressed oil obtained from date kernels

| | Percentage of |
|---|---|
| Fatty acid | total fatty acids |
| C_{12:0} | 8.0% |
| C_{14:0} | 6.3% |
| C_{16:0} | 21.8% |
| C_{18:2} | 10.0% |
| C_{18:1} | 45.6% |
| C_{18:0} | 8.3% |

These results indicate that oil can be obtained from date kernels by cold pressing, and that the yield obtainable by cold pressing (about 10-25%) compares favorably with those reported in the art for extraction utilizing organic solvents and/or more costly techniques [Mrabet et al., Foods 2020, 9:787].

These results further show that cold-pressed oil obtained from date kernels (generally a waste product) can serve as a source of relatively short fatty acids, such as lauric acid and/or myristic acid; e.g., as an alternative to more environmentally problematic (due to their tropical habitat) oil palm kernel oil and coconut oil.

These results further indicate that cold-pressed oil obtained from date kernels can serve as a complementary oil to the oil obtained from date offshoots (e.g., as described in Example 1 hereinabove), by including a different composition of free fatty acids, and that such a combined oil, which includes a desirable composition of fatty components, represents an alternative to currently available oil compositions that are produced, for example, from oil palm fruit (a source of palmitic acid), oil palm kernel and/or coconut (sources of shorter fatty acids) and combinations thereof.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-40. (canceled)

41. A method of preparing a vegetable oil, the method comprising pressing a non-fruit tissue of a date palm offshoot, to thereby obtain the vegetable oil.

42. The method of claim 41, wherein said non-fruit tissue is an agricultural waste material.

43. The method of claim 41, wherein at least a portion of said non-fruit tissue is meristematic tissue.

44. The method of claim 43, further comprising removal of non-meristematic tissue prior to said pressing.

45. The method of claim 41, wherein said pressing comprises cold pressing.

46. The method of claim 41, further comprising separating a polysaccharide-containing fraction from an oil-containing fraction subsequently to said pressing.

47. The method of claim 46, wherein said separating is effected by contacting a liquid obtained by said pressing with a water-immiscible solvent.

48. The method of claim 47, wherein said water-immiscible solvent is dichloromethane.

49. A composition comprising a polar phase and a nonpolar phase, the composition being obtained according to the method of claim 41, wherein said nonpolar phase comprises said vegetable oil, and said polar phase comprises polysaccharides.

50. A vegetable oil obtained according to the method of claim 41.

51. A polysaccharide-containing fraction obtained according to the method of claim 46.

52. An oil comprising esters of fatty acids, wherein at least 10 weight percent of said fatty acids are linolelaidic acid.

53. The oil of claim 52, wherein at least 20 weight percent of said fatty acids are palmitic acid.

54. The oil of claim 52, wherein at least 50 weight percent of said fatty acids are selected from the group consisting of palmitic acid and linolelaidic acid.

55. The oil of claim 52, wherein at least 5 weight percent of said fatty acids are stearic acid.

56. The oil of claim 52, wherein at least 70 weight percent of said fatty acids are selected from the group consisting of palmitic acid, stearic acid and linolelaidic acid.

57. The oil of claim 56, wherein no more than 10 weight percent of said fatty acids are oleic acid.

58. The oil of claim 57, wherein no more than 10 weight percent of said fatty acids are selected from the group consisting of oleic acid, lauric acid and myristic acid.

59. The oil of claim 52, wherein said esters of fatty acids comprise triglycerides.

60. The oil of claim 52, further comprising at least one phytosterol.

61. The oil of claim 60, wherein a total concentration of said esters of fatty acids and said at least one phytosterol is at least 90% of the oil by weight.

62. The oil of claim 52, further comprising dihydrouracil.

63. The oil of claim 52, comprising:
esters of palmitic acid, linolelaidic acid, stearic acid, oleic acid, linoleic acid and pentadecanoic acid;
β-sitosterol;
stigmasterol;
campesterol;
cholesterol;
cyclolanosterol; and
dihydrouracil.

64. A method of preparing a vegetable oil, the method comprising pressing date kernel tissue, to thereby obtain the vegetable oil.

65. The method of claim 64, wherein said pressing comprises cold pressing.

66. The method of claim 65, wherein said pressing is effected in a device with a rotating pole and an outlet configured for releasing said oil, wherein a diameter of said outlet is at least 5 mm.

67. A method of obtaining a vegetable oil characterized by:
at least one of:
(a) at least 5 weight percent of the fatty acids of the oil are lauric acid;
(b) at least 5 weight percent of the fatty acids of the oil are myristic acid; and
(c) at least 8 weight percent of the fatty acids of the oil are lauric acid or myristic acid,
as well as at least one of:
(i) at least 25 weight percent of the fatty acids of the oil are palmitic acid; and
(ii) no more than 30 weight percent of the fatty acids of the oil are oleic acid,
the method comprising combining an oil of claim 52 and a vegetable oil from date kernel tissue.

68. A method of obtaining a vegetable oil from agricultural waste material, the method comprising:
obtaining an oil from a non-fruit tissue of a date palm offshoot;
obtaining an oil from date kernel tissue; and
combining said oil from a non-fruit tissue of a date palm offshoot and said oil from date kernel tissue to obtain the vegetable oil characterized by:
a concentration of lauric acid which is at least 2 weight percent of the fatty acids of the vegetable oil;
a concentration of myristic acid which is at least 2 weight percent of the fatty acids of the vegetable oil;
a concentration of palmitic acid which is at least 20 weight percent of the fatty acids of the vegetable oil; and
a concentration of oleic acid which is no more than 35 weight percent of the fatty acids of the vegetable oil.

69. The method of claim 67, wherein said vegetable oil from date kernel tissue is obtained by pressing date kernel tissue.
